(19) United States
(12) Patent Application Publication
(10) Pub. No.: US 2024/0407952 A1
(43) Pub. Date: Dec. 12, 2024

(54) COLD WEATHER WELDING MASK

(71) Applicant: Divide Oilfield Consulting Ltd., Tomahawk (CA)

(72) Inventor: Bradley BOBEY, Tomahawk (CA)

(73) Assignee: Divide Oilfield Consulting Ltd., Tomahawk (CA)

(21) Appl. No.: 18/736,931

(22) Filed: Jun. 7, 2024

(30) Foreign Application Priority Data
Jun. 9, 2023 (CA) 3202741

(51) Int. Cl. A61F 9/06

(52) U.S. Cl. CPC A61F 9/061

(57) ABSTRACT

A cold weather welding mask has a face shield with a first aperture positioned such that a wearer can look through the first aperture. A head attachment is attachable to the face shield such that the face shield is supported on the wearer's head. A lens is secured within the first aperture and the lens corresponds in size to the first aperture. A heating element is attached to an interior surface of the lens. A controller is provided in electrical communication with the heating element and a power source. The controller is movable between an on position and an off position. In the on position, the controller directs an electrical current from the power source to the heating element. In the off position, the controller prevents electrical current from reaching the heating element.

### FIELD OF THE DISCLOSURE

The present application relates generally to a cold weather welding mask having a heated welding lens.

### BACKGROUND

This section provides background information to facilitate a better understanding of the various aspects of the invention. It should be understood that the statements in this section of this document are to be read in this light, and not as admissions of prior art.

Condensation buildup in welding masks and helmets is common when welding in cold temperatures. This can result in fogging that blocks a welder's vision. In an attempt to eliminate condensation, welders may alter their helmets and masks by cutting openings or drilling holes around the mouth area, however altered helmets are often not permissible on many work sites due to safety regulations.

Currently, forced air helmet fans are often used to help eliminate condensation buildup and fogging. These helmet fans direct air flow over the welder's face and helmet lens. The forced air helmet fans can help keep welders cool and reduce welding fumes around the welder's face. There are, however, downsides to the forced air helmet fans in that they generally only partially remove condensation, and they distribute air at ambient temperatures. When working outdoors in cold weather, the ambient air can be very cold and the forced air helmet fans can become uncomfortable for the welder. In addition, many welders do not like having air blowing through their helmets and masks.

### BRIEF SUMMARY

There is provided a cold weather welding mask that has a face shield with a first aperture positioned such that a wearer can look through the first aperture. A head attachment is attachable to the face shield so that the face shield can be supported on the wearer's head. A lens is secured within the first aperture and corresponds in size to the first aperture. A heating element is attached to an interior surface of the lens to heat the lens. A controller is provided in electrical communication with the heating element and a power source. The controller is movable between an on position and an off position. In the on position, the controller directs an electrical current from the power source to the heating element. When in the off position, the controller prevents electrical current from reaching the heating element.

In one embodiment, the power source is a battery. The battery may be a rechargeable battery or may need to be replaced once depleted.

In one embodiment, the controller is positioned on an interior surface of the face shield.

In another embodiment, the controller is positioned on the head attachment.

In a further embodiment, the controller is movable and may be positioned anywhere on the face shield, heat attachment, placed in a wearer's pocket, or clipped to a wearer's clothing.

In one embodiment, the head attachment is a headband. Face shield may be fixedly or hingedly attached to head attachment.

In another embodiment, the heating element is positioned adjacent at least one edge of the lens.

In one embodiment, the heating element is positioned adjacent an entire perimeter of the lens.

In one embodiment, the controller is adjustable to allow the heating element to be heated to at least two predetermined temperatures.

In one embodiment, the face shield includes a projecting portion that extends outward to provide an extended space between the wearer's head and the lens. The first aperture is positioned within the projecting portion.

In one embodiment, the lens is made up of a first lens, a second lens, and a third lens. The second lens is positioned between the first lens and the third lens.

In another embodiment, a spacer is positioned between at least one of the first lens and the second lens or the second lens and the third lens. The spacer creates an air pocket with the lens that provides additional protections to the wearer.

In one embodiment, the face shield includes a hingedly attached lens holder. The lens holder has a second aperture. The lens holder is movable between an upwards position and a downwards position. In the downwards position, the second aperture aligns with the first aperture. In the upwards position, the lens holder is raised such that the lens holder does not obstruct the wearer's view. At least one lens is positioned with the second aperture.

In one embodiment, at least one of the lenses positioned in the second aperture is a UV resistant lens.

In another embodiment, the UV resistant lens is positioned between a first clear lens and a second clear lens.

### BRIEF DESCRIPTION OF THE DRAWINGS

These and other features will become more apparent from the following description in which references are made to the following drawings, in which numerical references denote like parts. The drawings are for the purpose of illustration only and are not intended to in any way limit the scope of the invention to the particular embodiments shown.

FIG. 1 is a front elevation view of a cold weather welding mask.

FIG. 2 is a rear elevation view of a cold weather welding mask.

FIG. 3 is a rear perspective view of a cold weather welding mask.

FIG. 4 is a side elevation view, partially in section, of a cold weather welding mask.

FIG. 5 is a rear elevation view of a cold weather welding mask with the controller on the head attachment.

FIG. 6 is a rear elevation view of a cold weather welding mask with the controller.

FIG. 7 is an exploded view of the cold weather welding mask.

FIG. 8 is a perspective view of a lens combination.

FIG. 9 is a perspective view of a variation of the cold weather welding mask.

FIG. 10 is a side elevation view, partially in section, of the variation of the cold weather welding mask shown in FIG. 10.

### DETAILED DESCRIPTION OF THE PREFERRED EMBODIMENTS

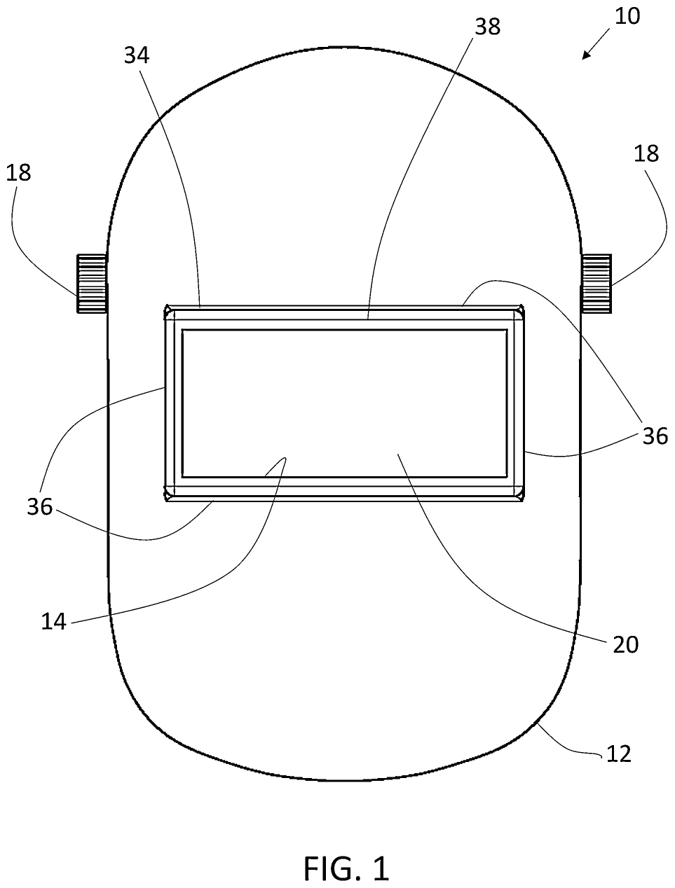

A cold weather welding mask, generally identified by reference numeral 10, will now be described with reference to FIG. 1 through FIG. 10.

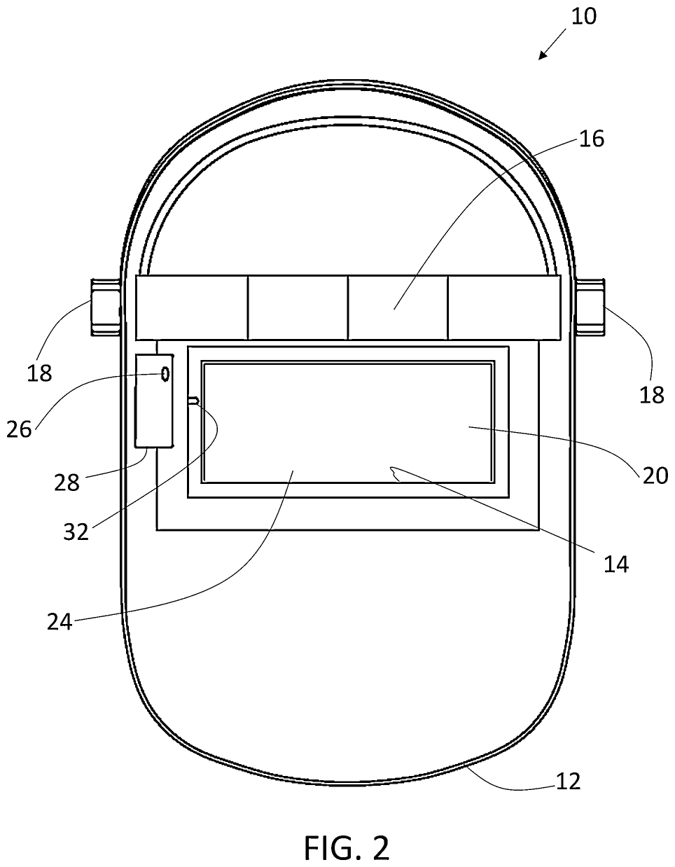

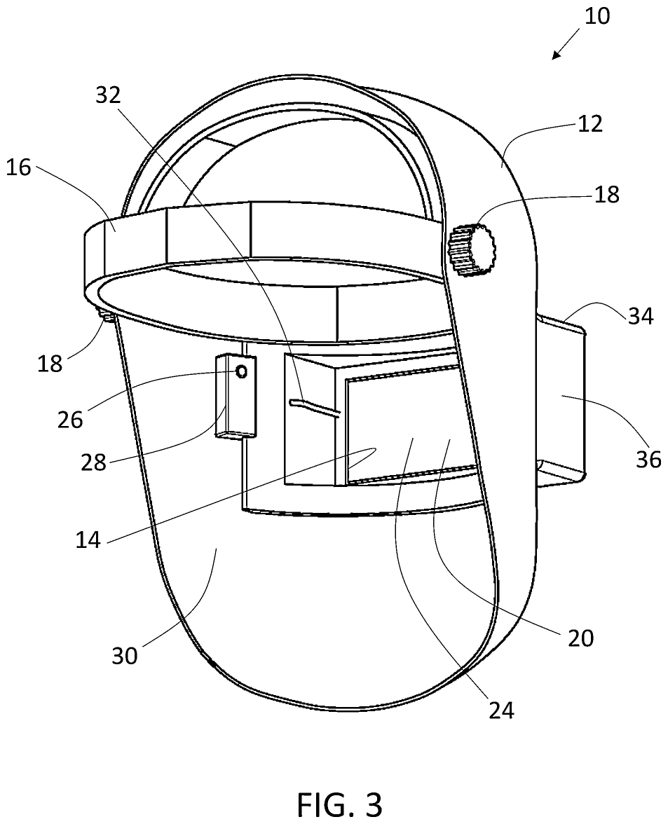

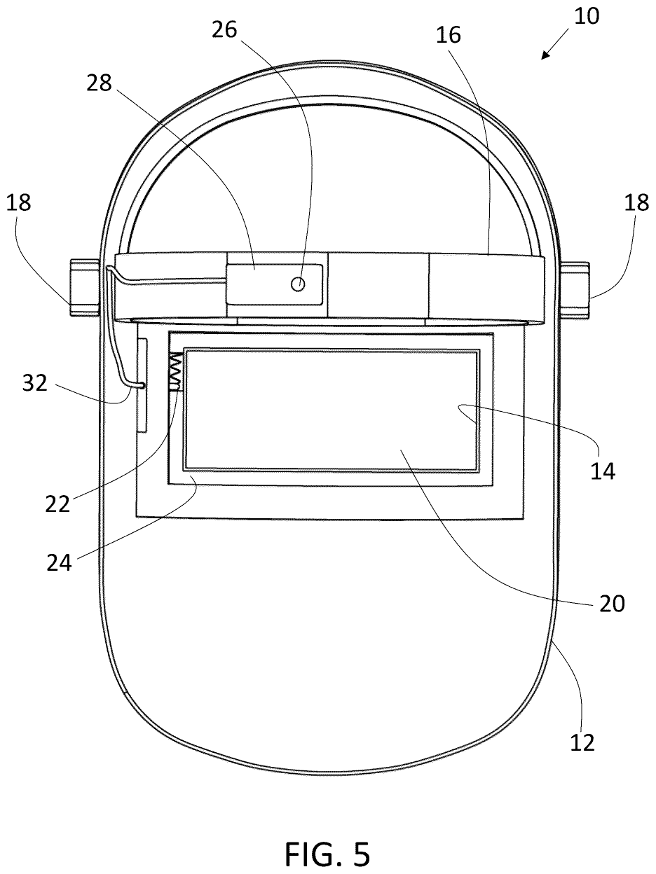

Referring to FIG. 1, a cold weather welding mask 10 has a face shield 12 with a first aperture 14 positioned such that a wearer can look through first aperture 14. Referring to FIG. 2, a head attachment 16 is attachable to face shield 12 such that face shield 12 is supported on the wearer's head. In the embodiment shown, head attachment 16 is a headband. It will be understood by a person skilled in the art that different sizes of head attachment 16 may be provided and head attachment 16 may be adjustable to allow for fitting to individual wearers. Face shield 12 may be fixedly or hingedly attached to head attachment 16. In the embodiment shown in FIG. 3, face shield 12 is hingedly attached to heat attachment 16 using fasteners 18. This allows a wearer to tilt face shield 12 upwards to uncover their face when not in use and tilt forward to cover the wearer's face when in use. A lens 20 is secured within first aperture 14. The size of lens 20 corresponds to the size of first aperture 14. Referring to FIG. 5, a heating element 22 is attached to an interior surface 24 of lens 20 to heat lens 20. It is important that heating element 22 be positioned such that it does not obstruct the wearer's vision. Obstructing the wearer's vision can result in poor quality work and injuries when the wearer is unable to see clearly. A controller 26 is provided in electrical communication with heating element 22 and a power source 28 such as a battery. Batteries may be rechargeable or single use and may be replaceable as needed. Controller 26 is movable between an on position and an off position. In the on position, controller 26 directs an electrical current from power source 28 to heating element 22. In the off position, controller 26 prevents electrical current from reaching heating elements 22. Controller 26 may be an on/off switch or may allow heating element 22 to be adjusted to different temperatures depending upon user preference and predetermined temperature settings.

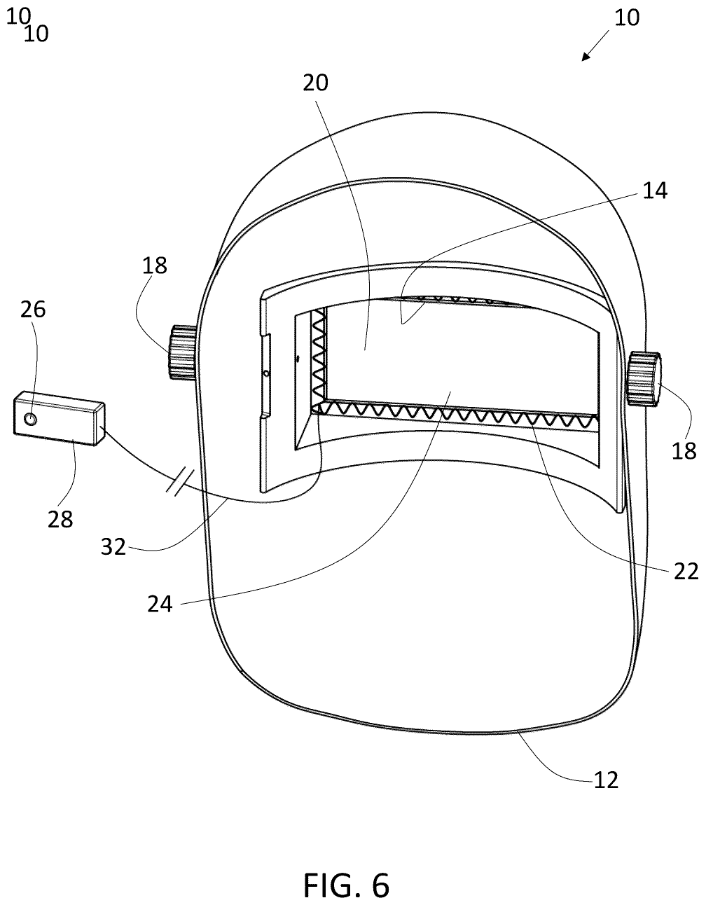

Controller 26 and power source 28 may be positioned in a number of locations depending upon a wearer's preference. In the embodiment shown in FIG. 3, controller 26 and power source 28 are positioned on an interior surface 30 of face shield 12. In the embodiment shown in FIG. 5, controller 26 and power source 28 are positioned on head attachment 16. In the embodiment shown in FIG. 6, controller 26 and power source 28 are positionable in a wearer's pocket or clipped on to a piece of clothing. It will be understood by a person skilled in the art that different lengths of electric wire/conduit 32 can be used to increase or decrease the distance controller 26 and power source 28 may be positioned from face shield 12. It is also possible for controller 26 to wirelessly control heating element 22.

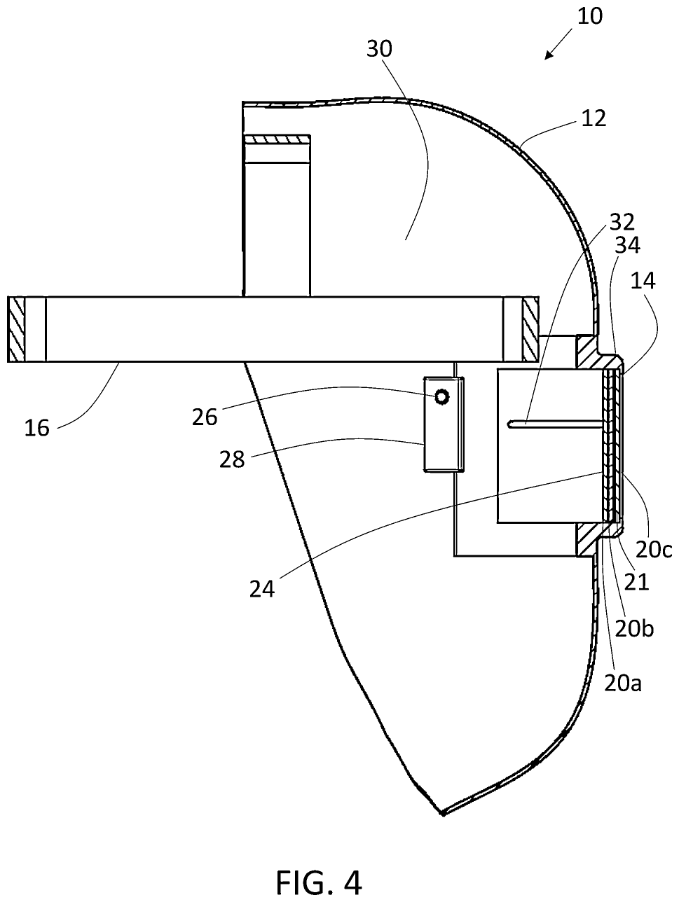

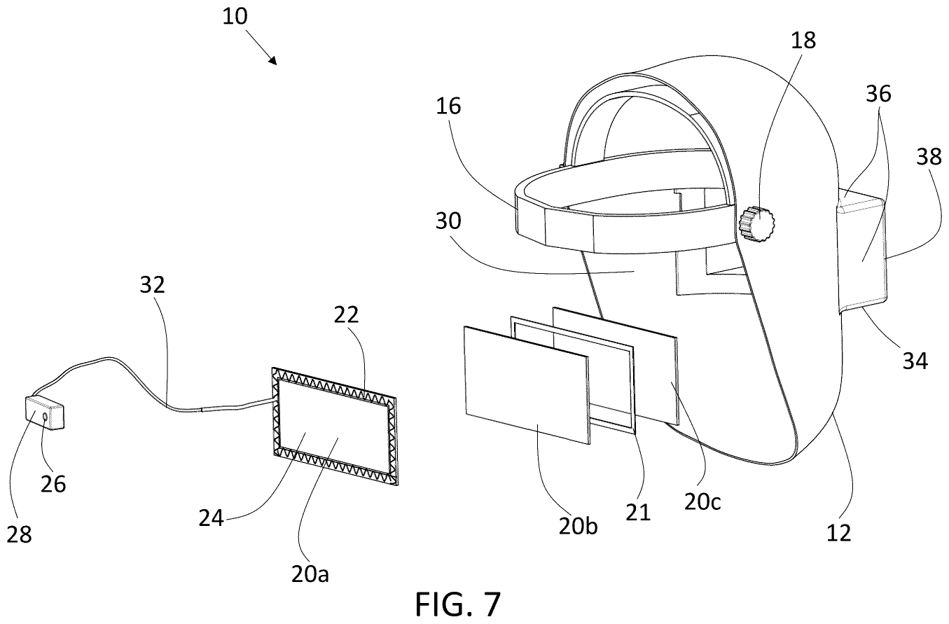

In the embodiment shown in FIG. 4 and FIG. 7, multiple lens are positioned in face shield 12 to provide additional protections and barriers to the wearer's eyes. It will be understood by a person skilled in the art that a single lens may be used, or multiple lenses may be used. When a single lens is used, that lens should be a UV resistant lens suitable for use for welding purposes. When multiple lenses are used, at least one of the lenses should be a UV resistant lens suitable for use for welding purposes. In the embodiment shown, heating element 22 is positioned on a first lens 20a. First lens 20a is a clear lens and is positioned such that it is the closest lens to a wearer's eyes. A second lens 20b is positioned adjacent first lens 20a. Second lens 20b is a UV resistant lens suitable for use for welding purposes. A third lens 20c is positioned adjacent second lens 20b such that second lens 20b is positioned between first lens 20a and third lens 20c. Third lens 20c is a clear lens and is the furthest lens from the wearer's eyes. A spacer 21 is provided between second lens 20b and third lens 20c to create an air space that provides additional protection to lens 20b in the event of damage to third lens 20c or excessive heat. It will be understood by a person skilled in the art that any one of first lens 20a, second lens 20b, third lens 30c, or a combination of these lens may be UV resistant lenses suitable for use for welding purposes. It will also be understood that any number of lenses may be used. It will further be understood that heating element 22 may be positioned on any first lens 20a, second lens 20b, or third lens 20c. It may be beneficial for heating element 22 to be positioned on first lens 20a as first lens 20a is the lens most likely to develop condensation.

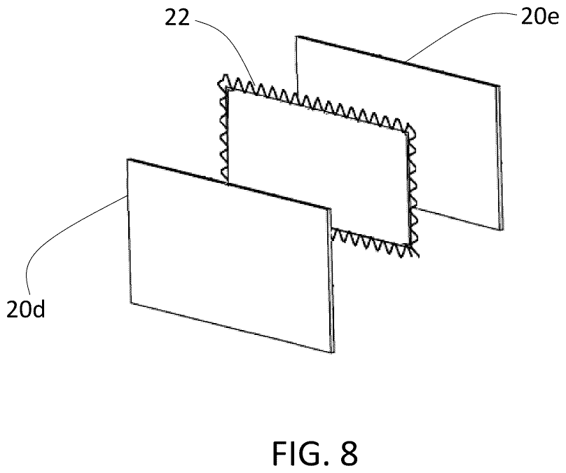

In the embodiment shown in FIG. 8, heating element 22 may be positioned between two clear lenses 20d and 20e, with lenses 20d and 20e sealed together to provide protection to heating element 22. It will be understood by a person skilled in the art that heating element may also be positioned between a clear lens and a UV resistant lens, or between two UV resistant lens. It will be understood by a person skilled in the art that embodiments where adjacent lens are not sealed together may also be used.

In the embodiment shown in FIG. 7, heating element 22 is positioned adjacent an entire perimeter of lens 20. A smaller heating element 22, shown in FIG. 5, may also be used and positioned adjacent at least one edge of lens 20. However, a smaller heating element 22 will be less effective in removing condensation than a heating element 22 positioned adjacent an entire perimeter of lens 20.

In the embodiments shown, face shield 12 has a projecting portion 34 that extends outwards away from a wearer's face to provide an extended space between the wearer's head and lens 20. This can be beneficial for wearer's who have glasses or wear additional protective eyewear. In the embodiments shown, projecting portion 34 is rectangular in shape, however it will be understood by a person skilled in the art that projecting portion 34 may be any other suitable shape. When projecting portion 34 is present, first aperture 14 is positioned within projecting portion 34. In the embodiment shown, projecting portion 34 has four peripheral side walls 36 and a front face 38. First aperture 14 is positioned in front face 38 such that lens is positioned within front face 38.

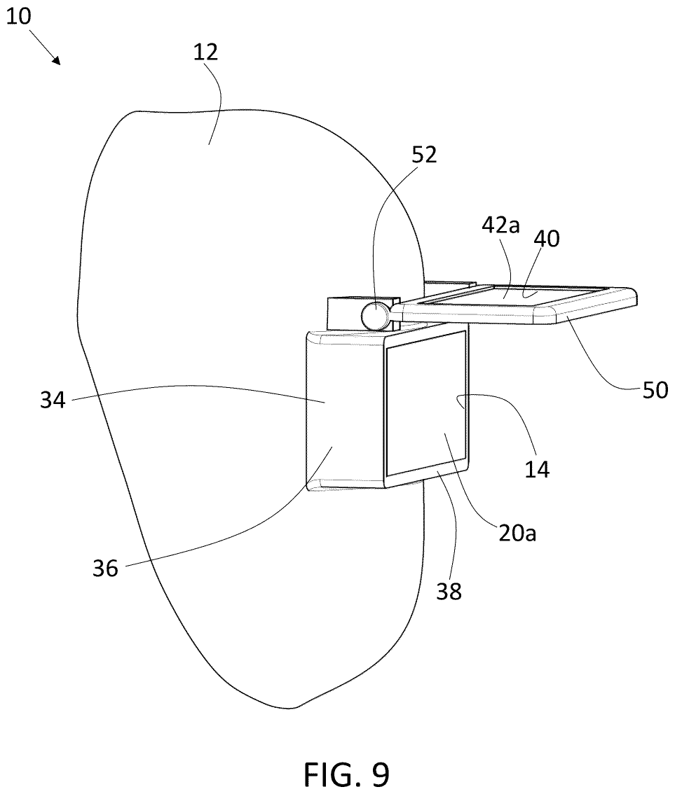

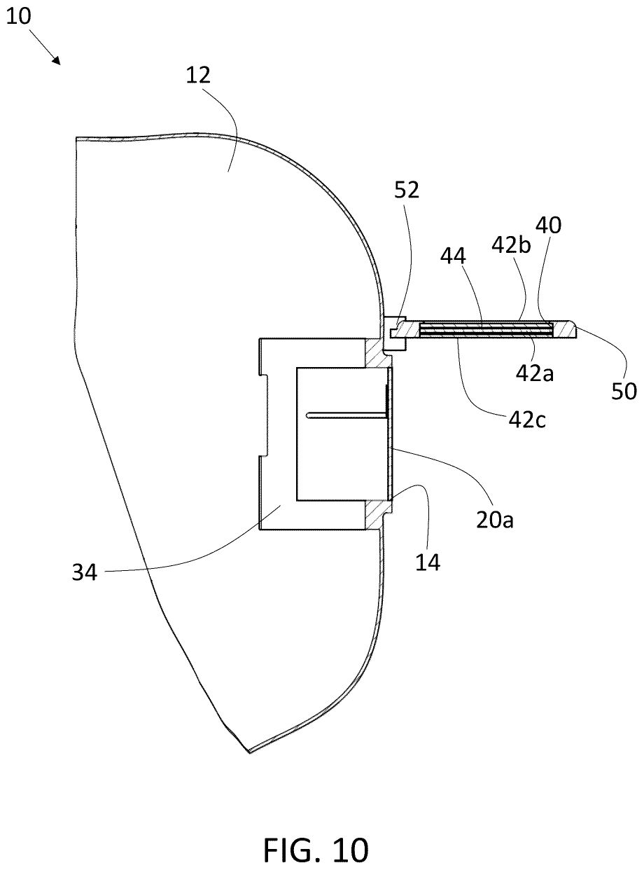

In the embodiment shown in FIG. 9 and FIG. 10, a variation of welding mask 10 is shown. In the embodiment shown, a lens holder 50 is hingedly attached to face shield 12 with hinge 52 to allow a wearer to flip lens holder 50 upwards relative to face shield 12. In this embodiment, first aperture 14 is positioned on face shield 12 such that a wearer can look through first aperture 14 and a clear lens 20a is fitted into first aperture 14. Lens holder 50 has a second aperture 40 that aligns with first aperture 14 when lens holder 50 is flipped downwards into contact with face shield 12. In the embodiment shown, lens holder 50 is flipped downwards to contact projecting portion 34 of face shield 12. Second aperture contains at least one UV resistant lens 42a. In the embodiment shown, second aperture 40 holds a UV resistant lens 42a positioned between a first clear lens 42b and a second clear lens 42c. A spacer 44 may be positioned between UV resistant lens 42a and second clear lens 42c. When lens holder 50 is flipped upwards relative to face shield 12, a wearer can view their work through clear lens 20a without the darkening effects of UV resistant lens 42a. When welding, a wearer can flip lens holder 50 downwards into contact with face shield 12 to protect their eyes with UV resistant lens 42a. It will be understood by a person skilled in the art that the shape of lens holder 50 may vary. Lens holder 50 may have side covers that allow lens holder 50 to envelope projecting portion 34.

It will be understood by a person skilled in the art that heating element 22 may be associated with any lens related to cold weather welding mask. It may be preferable for heating element 22 to be positioned on the lens positioned most closely to the wearer's face, however heating element 22 may also be placed on lens 42a, 42b, 42c positioned within aperture 40 of hingedly attached projecting portion 50 or multiple heating elements 22 may be used on different lenses.

Any use herein of any terms describing an interaction between elements is not meant to limit the interaction to direct interaction between the subject elements, and may also include indirect interaction between the elements such as through secondary or intermediary structure unless specifically stated otherwise.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent that changes may be made to the illustrative embodiments, while falling within the scope of the invention. As such, the scope of the following claims should not be limited by the preferred embodiments set forth in the examples and drawings described above, but should be given the broadest interpretation consistent with the description as a whole.

What is claimed is:

1. A cold weather welding mask, comprising:
a face shield having a first aperture positioned such that a wearer can look through the aperture;
a head attachment being attachable to the face shield such that the face shield is supported on the wearer's head;
a lens secured within the first aperture, the lens corresponding in size to the first aperture;
a heating element being attached to an interior surface of the lens;
a controller in electrical communication with the heating element and a power source, the controller being movable between an on position and an off position, in the on position the controller directs an electrical current from the power source to the heating element, when in the off position the controller prevents electrical current from reaching the heating element.

2. The cold weather welding mask of claim 1 wherein the power source is a battery.

3. The cold weather welding mask of claim 1 wherein the controller is positioned on an interior surface of the face shield.

4. The cold weather welding mask of claim 1 wherein the controller is positioned on the head attachment.

5. The cold weather welding mask of claim 1 wherein the controller is movable.

6. The cold weather welding mask of claim 1 wherein the head attachment is a headband.

7. The cold weather welding mask of claim 1 wherein the heating element is positioned adjacent at least one edge of the lens.

8. The cold weather welding mask of claim 1 wherein the heating element is positioned adjacent an entire perimeter of the lens.

9. The cold weather welding mask of claim 1 wherein the controller is adjustable to allow the heating element to be heated to at least two predetermined temperatures.

10. The cold weather welding mask of claim 1 wherein the face shield further includes a projecting portion, the aperture being positioned within the projecting portion, the projecting portion extending outwards to provide an extended space between the wearer's head and the lens.

11. The cold weather welding mask of claim 1 wherein the lens comprises a first lens, a second lens, and a third lens, the second lens being positioned between the first lens and the third lens.

12. The cold weather welding mask of claim 11 wherein a spacer is positioned between at least one of the first lens and the second lens or the second lens and the third lens, the spacer creating an air pocket within the lens.

13. The cold weather welding mask of claim 1 wherein the face shield has a hingedly attached lens holder, the lens holder having a second aperture, the lens holder being movable between an upwards position and a downwards position, when the lens holder is in the downwards position the second aperture aligns with the first aperture, in the upwards position the lens holder is raised such that the lens holder does not obstruct the wearer's view, at least one lens being positioned within the second aperture.

14. The cold weather welding mask of claim 13 wherein at least one of the lenses positioned with the second aperture being a UV resistant lens.

15. The cold weather welding mask of claim 14 wherein the UV resistant lens is positioned between a first clear lens and a second clear lens.
